(19) United States
(12) Patent Application Publication
(10) Pub. No.: US 2002/0120332 A1
(43) Pub. Date: Aug. 29, 2002

(54) Method and apparatus for securing external device to patient

(76) Inventors: Tom J. Law, Chandler, AZ (US); Albert A. Maltan, Stevenson Ranch, CA (US)

Correspondence Address: ADVANCED BIONICS CORPORATION, 12740 SAN FERNANDO ROAD, SYLMAR, CA, 91342, US

(21) Appl. No.: 10/006,031

(22) Filed: Dec. 3, 2001

Related U.S. Application Data
(60) Provisional application No. 60/257,722, filed on Dec. 21, 2000.

(51) Int. Cl. A61F 2/02; A61F 2/18

(52) U.S. Cl. 623/11.11; 623/10; 607/43

(57) ABSTRACT

An improved method for securing an external device of a implantable system, in cooperation with an implantable device of the implantable system, uses a medical-pressure-sensitive adhesive pad to attach the external device to skin. Both power and control signals may be transmitted transcutaneously from the external device to the implantable device. Efficient transmission of these signals requires that the external device be securely held in cooperation with the implantable device. The medical-pressure-sensitive adhesive pad acts as an interposing adhesive between the external device and the skin. One side of the pad provides adhesion to the back of the external device. The opposite side of the pad provides adhesion to the skin/hair. The pad includes a tab for easy removal of the pad from the external device. The adhesives have aggressive adhesion to the pad, so that upon the removal of the pad from the skin, and then the external device, all of the adhesive remains on the pad. Adhesives are available with several degrees of adherence to the skin, thus allowing the selection of the adhesive to suit the requirements of the user and to minimize trauma upon removal.

The present application claims the benefit of U.S. Provisional Application Serial No. 60/257,722, filed Dec. 21, 2000, which application is incorporated herein by reference.

### BACKGROUND OF THE INVENTION

The present invention relates to implantable medical device systems, and more particularly to a method for removably attaching an external device to a patient's skin, in cooperation with an implanted device. Many known implantable medical devices rely upon a Radio Frequency (RF) link between an external antenna in an external device and an implantable antenna in an implanted device, to provide power signals and/or control signals to the implanted device. Known Implantable Cochlear Stimulation (ICS) systems include a headpiece adapted to provide an electrical signal, representative of a transduced acoustic signal, to an implantable part of the ICS system. Similarly, Deep Brain Stimulation (DBS) systems, Spinal Cord Stimulation (SCS) systems, or other neurostimulation systems may utilize an external device to provide real-time control signals, updates to stimulation control parameters, to recharge an implantable battery, or to receive back transmitted device status or sensor data.

ICS systems are used to provide the sensation of hearing to those who are profoundly deaf, and for whom traditional hearing aids are of little or no assistance due to disease or damage to the middle ear or inner ear. An ICS system provides the sensation of hearing by applying electrical stimuli to the inside of the scala tympani duct of the cochlea, thereby directly stimulating the ganglion cells coupled to the auditory nerve. Once stimulated, such ganglion cells send nerve impulses to the brain through the auditory nerve, and the impulses are sensed in the brain as perceived sound

ICS systems typically include implantable and external components. The implantable components include an implantable pulse generator and an electrode array. The electrode array is inserted into the cochlea, and used to apply electrical stimuli to the auditory nerves. The external components include a power source, a microphone, a speech processor, and a headpiece. The microphone senses sound waves in conventional manner and transduces such sensed sound waves to an electrical signal. The electrical signal is then processed by the speech processor and converted into an appropriate control signal that is transmitted to the implantable receiver/stimulator. A representative cochlear implant system is described in U.S. Pat. No. 5,776,172, issued Jul. 7, 1998 for “Multichannel Implantable Cochlear Simulator,” incorporated herein by reference.

In operation, the power and control signals are transmitted to the implantable receiver/stimulator through the external antenna (a primary coil) located in the headpiece, and are received through the implantable antenna (a secondary coil) included within the implantable receiver/stimulator. In order to operate efficiently, i.e., in order for the headpiece to be able to transcutaneously (i.e., through the skin) transmit the control signal to the implantable receiver/stimulator, it is necessary that the primary coil in the headpiece be placed in close alignment with the secondary coil in the implantable receiver/stimulator.

The most common technique for retaining the headpiece of a transcutaneous-type cochlear implant system is the use of two magnets. One magnet resides in the implantable stimulator near the center of the secondary coil. The other magnet resides in the headpiece near the center of the primary coil. The use of magnets to retain the headpiece is very simple, effective and cosmetically attractive. However, there are several drawbacks to this method of retention. Some patients have thick skin flaps that increase the separation of the magnets and reduce their attracting force. Physically active children and adults have found magnetic headpiece retention insufficient. And, the internal magnet may interfere with MRI (magnetic resonance imaging) diagnosis.

Another method of retaining a head piece is by using VELCRO® pads as described in U.S. Pat. No. 5,545,191, dated Aug. 13, 1996 for “Method for Optimally positioning and Securing the External Unit of a Transcutaneous Transducer on the Skin of a Living Body.” The '191 patent describes several embodiments with different shaped VELCRO® pads. While the use of VELCRO® pads provides some advantages, it also has several drawbacks. The use of VELCRO® pads to retain the headpiece requires that either the hook or loop VELCRO® pad be semi-permanently attached to the skin with several adverse results: the long term attachment to the skin of such pad may cause irritation or itching, the pads attached to the skin may be snagged when the user is combing or brushing their hair, the user's hair may become tangled in the VELCRO® pads, and the extraction of tangled hair from the VELCRO® pads may cause significant discomfort. In addition to these ergonomic factors, the thickness of the VELCRO® pads may reduce the efficiency of the inductive coupling between the headpiece and implantable device. Efficient power use in ICS systems is a significant issue, thus it is important that the primary and secondary coils be as close as possible. Further, if either VELCRO® pad should become soiled, or if a user participates in work related or recreational activities that tend to soil or otherwise degrade the VELCRO® pads, frequent replacement of the VELCRO® pads may be required.

Although DBS systems, SCS systems, and other neurostimulation systems may not require a continuous real-time control signal from an external device, these systems still may require communication with an external device to obtain control parameter changes, to receive power signals to recharge an implanted battery, or to allow the implantable device to back transmit device status information or physiological measurements obtained from an implanted sensor. Therefore, an external device similar to the headpiece of an ICS system often requires attachment to the patient's skin.

Therefore, there is a need for a low cost, robust, cosmetically acceptable, and comfortable method and apparatus for attachment of an external antenna in cooperation with the antenna of an implantable stimulator.

### SUMMARY OF THE INVENTION

The present invention addresses the above and other needs by providing an improved method for securing an external device of an implantable system, in cooperation with an implantable device of the implantable system. The present invention uses a medical-grade pressure sensitive acrylate adhesive pad to attach the external device to the skin. The pad acts as an interposing adhesive between the external device and the skin. One side of the pad provides adhesion to the back of the external device. The opposite side of the pad provides adhesion to the skin/hair. The pad includes a tab for removal of the pad from the external device. The adhesives have aggressive adhesion to the pad, and are available with several degrees of adherence to the skin. Release liners are provided on both adhesive surfaces and include a release liner tab.

In accordance with one aspect of the invention, there is provided a thin medical-grade pressure sensitive acrylate adhesive pad for attachment of the external device to the skin. Known implantable systems inductively transmit power from the external device to the implantable device using a primary coil in the external device and a secondary coil in the implantable device. The efficiency of the inductive power transmission is reduced if the separation of the primary coil from and secondary coil is increased. The thin pad of the present invention provides for the external device to be almost directly against the skin, unlike known methods of attaching the external device using VELCRO® pads. Such efficient transmission of power is essential to miniature devices which have limited space for a battery.

It is a feature of the present invention to provide a secure means of attaching an external device. The present invention provides various adhesive strength. Such various strengths allow a user to select the adhesive strength required for the activity the user intends to participate in, thereby providing secure attachment of the external device for a variety of activities. Also, skin flaps may interfere with the effectiveness of magnetic retention means, but have no effect on the adhesive retention means of the present invention. When strong adhesion is not required, the user may select a weaker adhesive to minimize trauma upon removal.

It is a further feature of the invention to provide a low cost disposable pad with a tab for easy removal, and an adhesive with aggressive adhesion to the pad. Advantageously, the result of such aggressive adhesion is that when the pad is removed from the skin and the external device, the adhesive remains on the pad. Due to the low cost, there is no need to re-use pads, and nothing to wear out.

It is an additional feature of the invention to provide an external device retention means that may be used with or without a magnetic retention means, and is adaptable to present implantable systems. The pads may be used in a magnet-less implantable system, thus preventing any interference an implanted magnet would have on an MRI examination. The pads may also be used with an implantable system which includes a magnetic retention means, in which use the pads would augment the magnets during times of increased physical activity. Further, when used with a magnet-less implantable system, the space otherwise required by the magnets is made available for additional electronics, and the weight of the magnet is eliminated from the external device.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows a headpiece (HP) of an Implantable Cochlear Stimulation (ICS) system residing on the side of a user's head, in close alignment to an implantable device of the ICS system;

FIG. 2 depicts an adhesive attachment system for the headpiece; and

FIG. 2A shows a cross-sectional view of the attachment system taken along line 2A-2A of FIG. 2.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

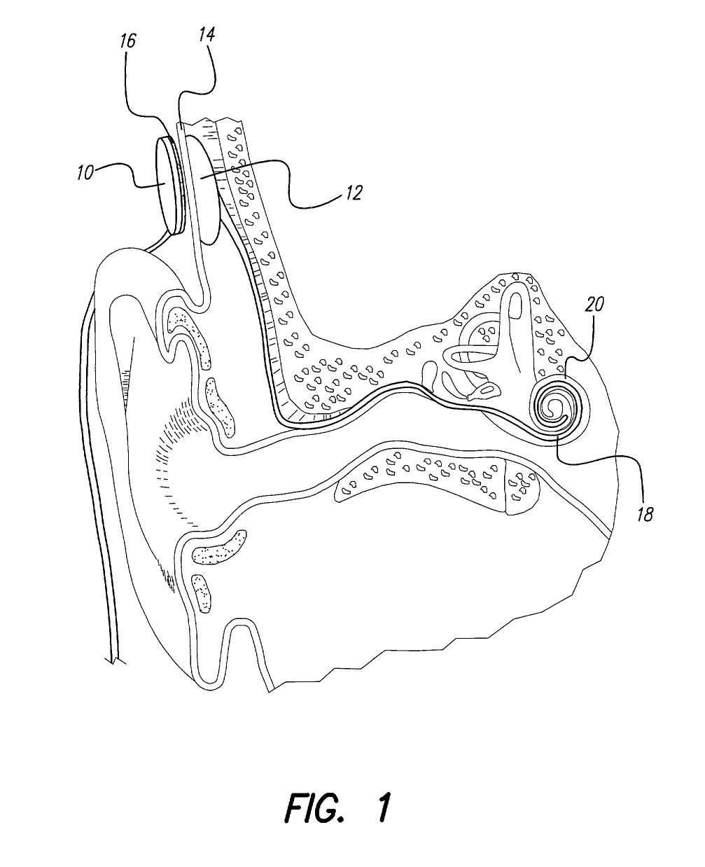

FIG. 1 shows an adhesive attachment system of the present invention attaching a headpiece (HP) 10 of an Implantable Cochlear Stimulation (ICS) system to the skin 14 of a user. An implantable device 12 is adjacent to the HP 10 on the opposite side of the skin 14. The HP 10 is held in place by a pad 16 residing between the HP 10 and the skin 14. The implantable device 12 is electrically connected to an electrode array 18 implanted in a cochlea 20. The HP 10 includes a primary coil of an inductive power transmission system, and the implantable device 12 includes a secondary coil of the inductive power transmission system.

Those skilled in the art will recognize that a similar or identical pad may be used to removably attach an external device of other implantable systems. An external device used to provide control signals (for example, adjust stimulation parameters), for a Spinal Cord Stimulation (SCS) system, a Deep Brain Stimulation (DBS), or other neurostimulation system may be held in place by such pad. Further, an external device used to provide power to directly power, or to recharge an energy storage device, for a neurostimulation system may be similarly held in place. These, and other applications of an adhesive pad used for securing an external device to a user's skin in cooperation with an implantable device, are intended to come within the scope of the present invention.

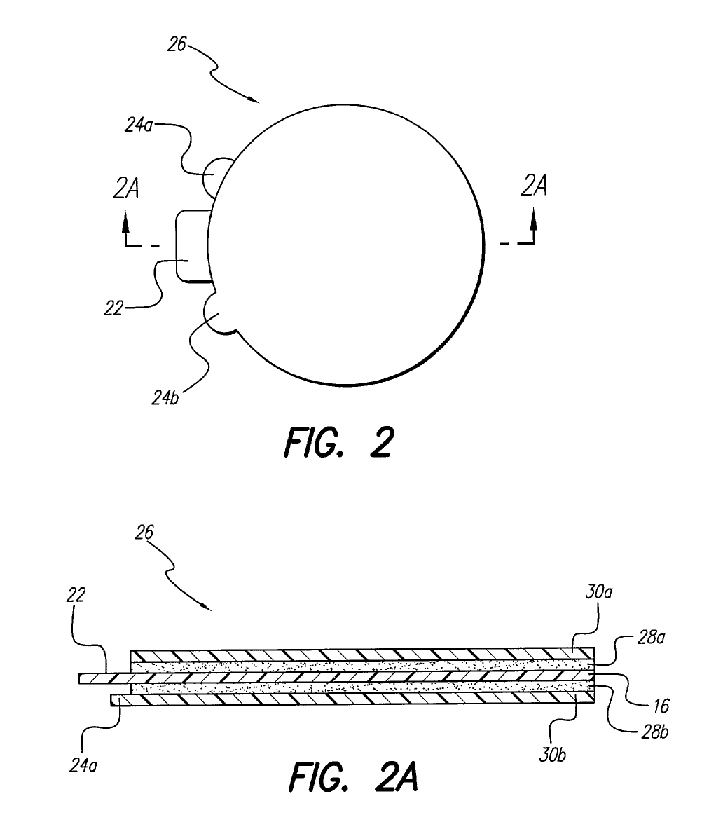

A side view of an adhesive attachment system 26 is shown in FIG. 2. The adhesive attachment system 26 for use with ICS systems is substantially round and is about one inch in diameter, preferably 1.060 inches in diameter. Three projections are apparent in the side view: a pad tab 22, a first liner tab 24a, and a second liner tab 24b. The utility of the tabs will be discussed in the following description of FIG. 2B.

Although the adhesive attachment system 26 shown in FIG. 2 is substantially round, adhesive attachment systems used with various types of implantable systems may include pads of various sizes and shapes suitable for the respective external device. In general, the shape of the adhesive attachment system matches the shape of the external device, but is not limited to the shape of the external device. In some applications, the adhesive attachment system may be a ring around the perimeter of the external device, allowing a central portion to directly contact the skin to place the external device and the internal device in close proximity. Those skilled in the art will recognize these various other shapes and sizes and other embodiments of the present invention, and these variations are intended to come within the scope of the present invention.

A cross-section taken along line 2A-2A of FIG. 2 is shown in FIG. 2A. The adhesive attachment system 26 comprises five layers. The top and bottom layers are a first release liner 30a and a second release liner 30b. The next layers are a first adhesive 28a residing on a first surface of the pad 16 and a second adhesive 28b residing on a second surface of the pad 16. The adhesives 28a and 28b are preferably medical-grade pressure-sensitive acrylate adhesives, and more preferably 3M® 1522 double coated medical tape. The center layer is the pad 16 (shown in use in FIG. 1 between the HP 10 and the skin 14.) The pad 16 is preferably a spunlaced polyester nonwoven fabric, and more preferably Avery Dennison MED 5707 white spunlaced fabric. The release liners 30a and 30b protect the adhesives 28a and 28b respectively. By covering the adhesives 28a and 28b prior to use, the adhesion of the adhesives 28a and 28b is preserved, and the adhesives 28a and 28b will not unintentionally become attached to some other material. The liner tabs 24a and 24b are provided to allow easy removal of the release liners 30a and 30b from the adhesives 28a and 28b respectively.

The adhesion of adhesives 28a and 28b may be selected to have aggressive adhesion to the pad 16, such that when the pad 16 is pulled away from the external device or the skin, the adhesives 28a and 28b will remain on the pad 16 and not become deposited on the external device or the skin. The adhesion of adhesive 28b may further be selected from a plurality of adhesives strengths (preferably 3) to allow the user to select the degree of adhesion of the pad 16 to the skin. The user may select a lessor degree of adhesion to allow trauma-free removal of the pad 16, or select a more aggressive level of adhesion when activities are planned that require stronger adhesion.

The degree of adhesion of the adhesive 28a to the HP 10 may be selected to ensure that the pad 16 remains attached to the HP 10 when the HP 10 is pulled away from the skin 14. The pad tab 22 is provided to allow the pad 16 to be easily pulled from the HP 10 after the HP 10 is pulled away from the skin 14. The pad 16 is preferably very thin (more preferably about 0.025 inches thick) to allow the primary coil to be as close as possible to the secondary coil.

Pressure Sensitive Adhesives (PSAs) of various adhesion strengths are known in the art. The adhesive strength of a PSA is measured by a known test called the 180 degree peel adhesion test. The test comprises applying a one half inch to one inch wide strip of the PSA to a flat metal surface, and then pulling the end of the PSA back 180 degrees over itself, and measuring the force required to peel the PSA at 12 inches per minute. The force is divided by the width of the PSA strip, to arrive at an adhesion measure with the units of pounds (or ounces) per inch of tape width. Alternatively, some PSAs are tested by a 90 degree test. Table 1 includes several medical grade PSAs and their adhesive strength.

TABLE 1
PSA Adhesion Strengths

| | | Adhesion |
|---|---|---|
| Manufacturer | Tape | (pounds/inch) |
| Avery | FM 2132 | 2.81 |
| Avery | Med 600 | 4.5 |
| Avery | Med 3044 | 3.6 |
| Avery | Med 2190H | 4.45 |
| Adhesion Research | ARCARE 7396 | 1.87 |
| Adhesion Research | ARCARE 8383 | 0.62 |
| Adhesion Research | ARCARE 8311 | 0.5 |
| Adhesion Research | ARCARE 8570 | 1.5 |
| 3M ® | 1522 | 0.87 |
| 3M ® | 9942 | N/A |
| 3M ® | 9874 | 0.75 |

The PSAs listed in Table 1 are manufactured by Avery Dennison Specialty Tape Division, 250 Chester Street, Painesville, Ohio 44077, Adhesion Research, Inc., 400 Seaks Run Road, Glen Rock, Pa. 17327, and 3M Medical Specialties Department, 3M Center, Building 275-5W-05, St. Paul, Minn. 55144

The availability of tabs and of various adhesive strengths is provided to enhance ease of use by the user. An adhesive attachment system according to the present invention could utilize any suitable adhesive of sufficient strength to attach the headpiece to the skin. Likewise, the release liners, and pad could be detached in the absence of tabs. Those skilled in the art will recognize various alternative embodiments of the present invention without tabs, or without the advantages of the adhesives described for FIG. 2A above, and these alternatives are intended to come within the scope of the present invention. Further, those skilled in the art will recognize alternative adhesives and pad materials. The scope of the present invention is intended to include embodiments utilizing other pad materials and adhesive materials used to similarly attach an external device to skin.

The adhesive attachment system of the present invention may be exercised alone, or in cooperation with a magnetic retention system. An existing ICS system may utilize magnets in the headpiece and in the implantable device to hold the headpiece in place, but may provide inadequate retention for some activities. In this case, the adhesive attachment system may be used to add to the retention provided by the magnets. In other applications, for example where a magnet is undesirable in the implantable device due to possible interference with MRI examinations, the adhesive attachment system may be the only headpiece retention method utilized. Those skilled on the art will recognize other external device retention means, and the cooperation of the adhesive attachment system with those other retention means is intended to come within the scope of the present invention.

By way of example, an adhesion attachment system for a headpiece of an ICS system is constructed in accordance with the invention by making a substantially round pad from MED 5707 white spunlaced fabric having approximate diameter of 1.060 inches. The pad preferably includes a pad tab to allow easy removal. A first adhesive comprising 3M® 1522 is placed on a first surface of the pad substantially covering the first surface, with a first release liner (preferably including a liner tab) facing away from the pad. A second adhesive comprising 3M® 1522 is placed on a second surface of the pad substantially covering the second surface, with a second release liner (preferably including a liner tab) facing away from the pad. When the adhesion attachment system is used, the first release liner is pulled off of the first adhesive, and the sticky surface of the exposed adhesive is pressed against the head piece of the ICS system. The second release liner is pulled off of the second adhesive, and the exposed sticky surface is pressed against the skin of the user, thereby holding the headpiece in its desired location.

In some instances (i.e., when strenuous physical activity is anticipated) a user may prefer a stronger adhesive between the pad and the user's skin. In this case, the adhesion attachment system for a headpiece of an ICS system is constructed as described above, except the 3M® 1522 used as the second adhesive is replaced by a stronger adhesive, e.g., Adhesion Research ARCARE 7396. The ARCARE 7396 has more than twice the adhesion of the 3M® 1522, thus providing a stronger bond between the pad and the user's skin.

While the invention herein disclosed has been described by means of specific embodiments and applications thereof, numerous modifications and variations could be made thereto by those skilled in the art without departing from the scope of the invention set forth in the claims.

What is claimed is:

1. An adhesive attachment system for a external device of an implantable system, the adhesive attachment system comprising:
a pad with a first surface and a second surface;
a first adhesive residing on the first surface of the pad; and
a second adhesive residing on the second surface of the pad;
wherein the pad it removably attachable to the external device by the first adhesive, and the pad is removably attachable to skin of a user by the second adhesive.

2. The adhesive attachment system of claim 1 wherein the implantable system includes an implantable device, and wherein the pad is adapted to hold the external device adjacent to the implantable device.

3. The adhesive attachment system of claim 1 wherein the first adhesive and the second adhesive have aggressive adhesion to the pad, wherein the first adhesive remains on the pad when the pad is removed from the external device, and wherein the second adhesive remains on the pad when the pad is removed from the skin.

4. The adhesive attachment system of claim 1 wherein the first adhesive has a more aggressive adhesion to the external device than the second adhesive has to the skin, wherein the pad remains attached to the external device when the external device is pulled away from the skin.

5. The adhesive attachment system of claim 1 further comprising a pad tab, wherein pad tab may be pulled to remove the pad from the external device.

6. The adhesive attachment system of claim 1 further comprising a first release liner and a second release liner, wherein the first release liner removably resides on the first adhesive and the second release liner removably resides on the second adhesive.

7. The adhesive attachment system of claim 6 wherein the first release liner includes a first liner tab, and the second release liner includes a second liner tab, and wherein the first release liner is removed from the first adhesive by pulling on the first line tab, and the second release liner is removed from the second adhesive by pulling on the second line tab.

8. The adhesive attachment system of claim 1 wherein the second adhesive comprises a plurality of second adhesives with different adhesion strengths, wherein one of the plurality of second adhesives may be selected by the user to provide the level of adhesion strength suitable for the level of activity anticipated by the user.

9. The adhesive attachment system of claim 8 wherein the plurality of second adhesives comprises adhesives of three different adhesive strengths.

10. The adhesive attachment system of claim 8 wherein the first adhesive comprises a plurality of first adhesives with different adhesion strengths, and wherein one of the plurality of first adhesives may be selected so that when the external device is pulled away from the skin, the pad will remain attached to the external device.

11. The adhesive attachment system of claim 1 wherein the pad is thin.

12. The adhesive attachment system of claim 11 wherein the pad is about 0.025 inches thick.

13. The adhesive attachment system of claim 1 wherein the adhesive attachment system is adapted to act as the sole means used to attach the external device to the skin.

14. The adhesive attachment system of claim 1 wherein the adhesive attachment system is used in cooperation with a magnet means for attaching the external device to the skin.

15. The adhesive attachment system of claim 1 wherein the pad is substantially flat and substantially round, and wherein the pad is about 1 inch in diameter.

16. The adhesive attachment system of claim 1 wherein the pad is 1.060 inches in diameter.

17. The adhesive attachment system of claim 1 wherein the implantable system is an Implantable Cochlear Stimulation (ICS) system.

18. A method for removably attaching an external device of an implantable system to skin, wherein the external device is in cooperation with an implantable device of the implantable system, comprising:
providing an adhesive attachment system, wherein the adhesive attachment system comprises a pad with a first surface and a second surface, wherein a first adhesive resides on the first surface and a second adhesive resides on the second surface;
pressing the first surface against the external device to removably attach the pad to the external device; and
pressing the second surface against the skin to removably attach the external device to the skin.

19. The method of claim 18 wherein the adhesive attachment system further includes a first release liner and a second release liner, wherein the first release liner removably resides over the first adhesive and the second release liner removably resides over the second adhesive, and wherein the method further includes:
removing the first release liner before pressing the first surface against the external device; and
removing the second release liner before pressing the second surface against the skin.

20. The method of claim 19 wherein the first release liner includes a first liner tab, and wherein the second release liner includes a second liner tab, wherein:
removing the first release liner comprises pulling on the first liner tab to remove the first release liner; and
removing the second release liner comprises pulling on the second liner tab to remove the second release liner.

21. The method of claim 18 wherein the method further includes pulling on the external device to remove the external device and pad from the skin.

22. The method of claim 18 wherein the pad includes a pad tab, and wherein the method further includes pulling the pad tab to remove the pad from the external device.

23. An adhesive attachment system for a external device of an implantable system, the adhesive attachment system comprising:
a pad with a first surface and a second surface;
a first adhesive residing on the first surface; and
a second adhesive residing on the second surface;
wherein the pad it removably attachable to the external device by the first adhesive, and the pad is removably attachable to skin of a user by the second adhesive; and
wherein the adhesive attachment system attaches the external device to the skin to provide for the cooperation of the external device with an implantable device.

24. The system of claim 23 wherein the implantable system is a Spinal Cord Stimulation (SCS) system.

25. The system of claim 23 wherein the implantable system is a Deep Brain Stimulation (DBS) system.

26. The system of claim 23 wherein the cooperation of the external device with the implantable device includes providing power to the implantable device.

27. The system of claim 23 wherein the cooperation of the external device with the implantable device includes providing control signals to the implantable device.
